(19) United States
(12) Patent Application Publication
(10) Pub. No.: US 2009/0247908 A1
(43) Pub. Date: Oct. 1, 2009

(54) MEASURING DEVICE

(76) Inventor: Angus Buchan Gordon, London (GB)

Correspondence Address: MERCHANT & GOULD PC, P.O. BOX 2903, MINNEAPOLIS, MN, 55402-0903, US

(21) Appl. No.: 12/441,801

(22) PCT Filed: Sep. 17, 2007
(86) PCT No.: PCT/GB07/03526

§ 371 Date: Apr. 9, 2009

(30) Foreign Application Priority Data
Sep. 18, 2006 (GB) 0618342.0

(51) Int. Cl. A61B 5/103

(52) U.S. Cl. 600/587

(57) ABSTRACT

The present invention relates to a tumour volume measuring device, comprising: a measurer for measuring at least one diameter of a tumour; a keyboard for inputting the at least one diameter of the tumour into the device; a processor linked to a keyboard, and a display which displays the calculated volume.

The present invention relates to a measuring device. In particular, the present invention relates to a measuring device for measuring and calculating the volume of an object. More particularly, the present invention relates to a tumour volume measuring device which, in use, can be used to measure and calculate the volume of a tumour. The present invention also relates to a calculator specifically adapted so that it may be used to calculate the volume of a tumour mass, malignant or benign, or other body mass. Furthermore, the present invention also provides a method for calculating the volume of a body mass, such as a tumour.

As will be appreciated by medical practitioners, it is important to be able to assess the rate at which a tumour is growing or regressing within a patient's body so that they are suitably placed to make an estimate of tumour activity.

Also, it will be appreciated by practitioners that such measurements also enable them to assess the effectiveness of a course of treatment and its effect on the tumour being treated, that is, by way of reference to changes in size.

Tumour size can be monitored by carrying out an appropriate scan, including mammogram, ultrasound, CT, MRI and PET scans of the tumour, and then, calculating the size of the tumour based on readings taken from the recorded image of the tumour from the scan.

With a view to tracking the growth of a tumour, some practitioners rely on differences in diameter as being representative of tumour progression or regression. However, such a one dimensional assessment can underestimate the actual rate of growth or remission of a tumour.

In order to gain a more accurate indication as to the progression of a tumour disease condition, it is better to measure changes in volume. This is reflected by the following example:

If, based on a reading taking from an image of a tumour represented by a scan, a tumour diameter had shrunk from 1.3 to 1.0 centimetres, one could conclude that this degree of remission is indicative of a minor response to treatment; however, if you consider the actual change in volume of the tumour it will be apparent that the volume of tumour has more than halved from 1.15 cm³ to 0.52 cm^{3.}

Therefore, and as will be appreciated, volume assessment of tumours has significant applications in clinical, radiological and histopathological applications.

At present, there is nothing in the art which allows a medical practitioner to readily and reliably measure and calculate the volume of a tumour mass.

It is an object of the present invention to at least address the problem identified above by the provision of a tumour volume measuring device or calculator, which readily and accurately enables a medical practitioner to calculate the volume of a tumour mass thereby enabling him/her to have more accurate clinical information.

According to a first aspect of the present invention there is a provided a tumour volume measuring device, comprising

a measurer for measuring at least one diameter of a tumour,

a keyboard for inputting the at least one diameter of the tumour into the device,

a processor linked to the keyboard, and

a display which can display the calculated volume.

As will be appreciated, a tumour volume measuring device in accordance with the present invention will enable a medical practitioner to readily, easily and accurately calculate the volume of a tumour so that he or she is positioned to make a suitable estimate based on the degree of growth or response of a tumour, that is, based on readings or measurements taken from a scan of the tumour.

Preferably, the measurer for measuring at least one diameter of a tumour is provided by a side of the device, which is graduated in units of length, further preferably, the units of length are centimetres and millimetres. It is to be appreciated that any suitable unit of length can form the basis of the graduations.

Advantageously, the device is rectangular or substantially rectangular in shape. This has the advantage in that the device is easy to hold and can be stored easily about the body of a person, for example, in a pocket.

More preferably, the device is portable and configured to be hand held. This has the advantage in that the device is easy to use.

Further preferably, the keys of the keyboard are arranged in at least two banks, each bank including keys relating to a distinct part of the calculation process. This has the advantage in that it reduces input error in that since each bank includes keys relating to a different or distinct part of the volume calculation procedure, the user is less likely to input data incorrectly by pressing a key in the wrong bank.

As medical practitioners will be aware, there are essentially three different tumour shapes, namely, spherical, ellipsoid and irregularly shaped tumours; the latter also referred to in the art as “date” shaped. It is worthy of note that up until now there is no known equation for accurately calculating the volume of an irregularly shaped tumour. In this regard, the present inventors have devised the formula below that enables the volume of an irregularly shaped to be accurately calculated. Such formula is represented as follows:

Volume=4/3πA.B.C

in which A is the radius of the longest axis/diameter, B is the radius of the next longest diameter and C is the radius of the shortest diameter/axis. By the use of the above equation, the volume of an irregularly shaped tumour can be accurately measured. Preferably the constant, 4/3×π, utilised in the above equation is calculated to six decimal places, namely, 4.188790. This reduces inaccuracies due to propagation of errors.

In a second aspect of the present invention there is provided a method for calculating the volume of an irregularly shaped body mass, such as a tumour, the method comprising the steps of:

measuring the three longest diameters of the irregularly shaped body mass; and then

calculating the volume using the following formula:

Volume=4/3πA.B.C

in which A is the radius of the longest axis/diameter, B is the radius of the next longest diameter/axis and C is the radius of the shortest diameter/axis

As stated above, by utilising the above formula, the volume of an irregularly shaped body mass, such as a tumour, benign or malignant, can be accurately calculated.

In yet another aspect of the present invention, there is provided a calculator for use in calculating the volume of a tumour or other body mass, the calculator being provided with a key which represents the constant, 4/3π such that the volume of a tumour or other body mass can be readily calculated utilising the calculator.

As will be appreciated from the disclosure below such a calculator is specifically adapted to calculate the volume of all tumour shapes, be it, spherically shaped, ellipsoid in shape or irregularly shaped.

Preferably, the constant 4/3π is pre-calculated to six decimal places i.e. when pressed, the constant key will input the figure 4.188790 into the calculator. This increases the accuracy of the volume calculated.

A non-limiting embodiment of a tumour measuring device and calculator of the present invention will be now be described by way of reference to the accompanying drawing in which:

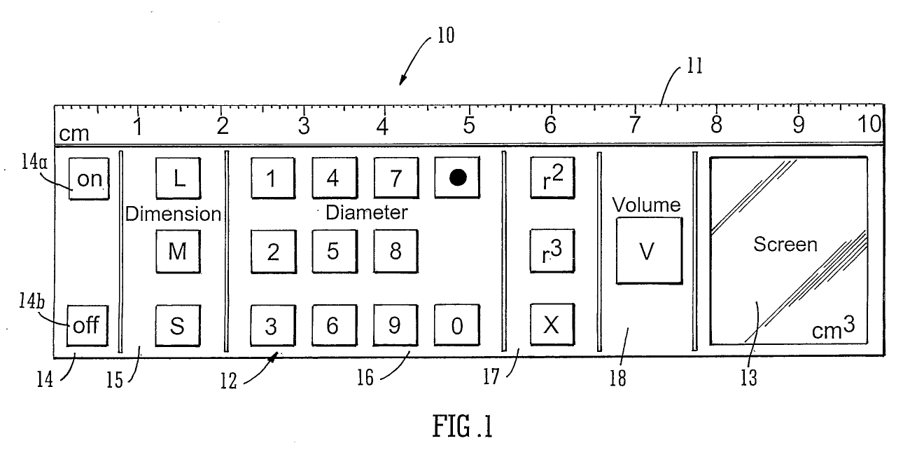

FIG. 1 is a plan view of one embodiment of a tumour volume measuring device or calculator in accordance with the present invention.

As illustrated in FIG. 1, a tumour volume measuring device and calculator (10) in accordance with the present invention is substantially rectangular in shape and includes one side (11), which is graduated in metric units. In the illustrated embodiment, the graduated side portion (11) is graduated in centimetres and millimetres.

The tumour volume measuring device (10) further includes a keyboard (generally represented by 12) such that the measurements taken by the use of the measuring portion (11) can be keyed into the device (10) so that the volume of a tumour or other body mass can be accurately calculated by a suitable processor (not shown) within the device (10) and displayed on the screen (13), which may be an LCD screen.

With further reference to the keyboard (12), it will be noted that it includes a number of separate banks of keys (represented generally by 14, 15, 16, 17 and 18).

The device also includes “off” and “on” buttons (14a and 14b respectively)

The use of the tumour volume measuring device (10) to measure volume size will now be explained further by way of reference to FIG. 1.

As touched upon above, and as medical practitioners will be aware, there are essentially three different types of tumour shapes, namely, spherical tumours, ellipsoid tumours and irregularly shaped tumours, which are referred to in the art as “date” shaped tumours.

In the event that a medical practitioner is provided with an image of a tumour mass, for example, an image obtained by the use of a mammogram scan, an ultrasound scan, a CT scan, and MRI scan and/or a PET scan, the device (10), being portable, can be readily placed over the image such that the measuring portion (11) can be utilised to take the appropriate measurement(s) depending on the shape of the tumour; the volume of which is calculated.

### A Spherical or Substantially Spherical Tumour

In the event that the tumour is spherical or substantially spherical, then all that is required of the practitioner or user of the device (10) is to measure the diameter of the tumour by utilising the graduated portion (11) of the tumour volume measuring device (10).

Once the diameter has been measured, the device (10) can be switched on by way of the “on” button (14).

Once the device (10) is switched “on” via button (14a), that is, if it is not “on” already, the user then keys the diameter measured by way of bank of keys (16) into the device (10). The figure entered will be displayed on the screen (13). Subsequent to entering a diameter, the user, by pressing the appropriate key in bank (15), then identifies to the device (10) that this is the longest diameter being measured. In the present illustrated embodiment, the key designated “L” is pressed subsequent to entering the longest diameter, the key designated “M” is pressed subsequent to entering the second longest diameter and the key designated “S” is pressed subsequent to entering the shortest diameter or the next longest diameter, that is, relative to the first two diameters measured. As will be appreciated, the amount of diameters entered into the device will depend on the initial assessment of the shape of the tumour (for which see below).

In the present instance, since the tumour being measured is a spherical mass, then only one diameter need be measured and the volume can be calculated utilising the following formula:

Volume=4/3πA³

in which “A” is the radius of the diameter measured.

Once the diameter has been keyed in by way of bank of keys (16), the user then presses “L” in bank (15) thereby signifying to the processor within the device (10) that this is the longest diameter being measured. On doing so, the diameter which has been keyed in will be automatically halved thereby displaying the radius “A” on the screen (13). The user, then presses the “r³” key in bank (17) such that the radius is automatically cubed and displayed on screen (13). The user then keys the multiplication key represented by “×” also in bank (17) and then the constant key represented by “V”, which is the sum of 4/3π, in bank (18) such that the calculated volume of the spherical mass or substantially spherical mass is displayed on the screen (13).

After use, the device (10) can be switched off by way of its “off” button (14b).

### An Ellipsoid Mass

The volume of an ellipsoid mass can be calculated by using the following formula:

Volume=4/3πAB²

in which A is the radius of the major axis (longest diameter) and B is the radius of the minor axis.

If the tumour to be measured is assessed to be ellipsoid in shape, then the user will measure the two appropriate diameters by way of the graduated portion (11) of the device (10), that is, by placing the graduated portion over the image of the tumour—shown by the scan.

Once the diameters have been measured, the user will key in the longest diameter first by way of keys in bank (16) into the device. Subsequent to doing so, the user will then press the key designated “L” in bank (15), which will result in the first, longest radius being displayed on screen (13). The user will then press the multiplication key “×” and then key in the second diameter measured, and then press the key designated “M” signifying to the processor that this is the second longest diameter. The processor will then automatically half the diameter such that the radius of the second longest diameter will be displayed on the screen (13). By subsequently pressing the key “r²” in bank 17, the second longest radius will be automatically squared. The user then presses the multiplication key “×” again such that the sum of AB² is calculated and displayed on the screen (13). Then, on pressing the constant key “V” in bank (18), the figure calculated for AB² is multiplied by the constant 4/3π resulting in the calculated volume of the ellipsoid tumour mass being displayed on the screen (13).

Date Shaped Tumours i.e. Irregular Shaped Tumours

In the case of irregular shaped tumours, there will be three separate diameter measurements available, which will provide greater accuracy. In this regard, the volume of this clinically relevant shape can be calculated using the following formula devised by the present inventor:

Volume=4/3πA.B.C

in which A is the radius of the longest axis/diameter, B is the radius of the next longest diameter and C is the radius of the shortest diameter/axis.

Once the appropriate diameter measurements of the various axis have been measured utilising the graduated portion (11), then the device (10) can be switched on via the “on” button (4a). The first diameter i.e. the longest diameter is keyed in by way of keys in bank (16) and then key “L” is pressed thereby automatically displaying the radius (“A”). The multiplication key “×” is then keyed, and then the second longest diameter is then keyed in; once again by way of the keys in bank (16). The key designated “M” in bank (15) is then pressed so that the radius of the second longest diameter is calculated and subsequently displayed on the screen (13). The multiplication key “×” is then keyed again and then the third longest diameter is keyed in as above, and this time the key designated “S” in bank (15) is subsequently pushed thereby resulting in the shortest radii being calculated. Then, by keying the multiplication key “×” again, the amount of A×B×C is displayed on the screen (13). Finally, and in accordance with the equation above, the user then only has to press the constant key designated “V” in bank (18) such that the sum of A×B×C is multiplied by the constant 4/3π thereby resulting in the calculated volume being displayed on the screen (13).

As will be appreciated from the above, the present application provides a device that readily enables the volume of a tumour mass to be calculated.

As will also be appreciated, as the keys are arranged in separate banks the user is less likely to press the wrong key resulting in error. Put another way, as each bank relates to a separate part of the calculation process, the user on carrying out the calculation sequentially is less likely to press a key in the wrong bank.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A tumour volume measuring device, comprising
a measurer for measuring at least one diameter of a tumour,
a keyboard for inputting the at least one diameter of the tumour into the device,
a processor linked to the keyboard, and
a display which displays the calculated volume.

2. The device of claim 1 in which the measurer is provided by a side of the device, which is graduated in units of length.

3. The device of claim 2 wherein the graduations are centimetres.

4. The device of claim 1, wherein the device is rectangular or substantially rectangular in shape.

5. The device of claim 1, wherein the device is portable and configured to be hand held.

6. The device of claim 1, wherein the keys of the keyboard are arranged in at least two banks, each bank including keys relating to a distinct or different part of the calculation process.

7. The device of claim 1, wherein the display is an LCD screen.

8. (canceled)

9. The use of the device of claim 1, wherein to calculate the volume of a tumour or other body mass based on measurements taken from an image of the tumour or body mass.

10. (canceled)

11. A method for calculating the volume of an irregularly shaped body mass, such as a tumour, the method comprising the steps of: measuring the three longest diameters of the irregularly shaped body mass; and then calculating the volume using the following formula:
Volume=4/3πA.B.C
in which A is the radius of the longest axis/diameter, B is the radius of the next longest diameter/axis and C is the radius of the shortest diameter/axis

12. The method of claim 11, wherein the three longest diameters of the irregularly shaped body mass are measured from an image of the body mass.

13. The method of claim 11, wherein the constant 4/3π is calculated to six decimal places.

14. The method of claim 1, wherein the volume of the irregularly shaped body mass is calculated using the tumour measuring device.

15.-16. (canceled)

17. A calculator for use in calculating the volume of a tumour or other body mass, the calculator being provided with a key which represents the constant, 4/3π such that the volume of a tumour or other body mass can be readily calculated utilising the calculator.

18. The calculator of claim 17, wherein the constant 4/3π is pre-calculated to six decimal places such that when the constant key is pressed, the constant key will input the figure 4.188790 into the calculator.

19. The calculator of claim 17, wherein the calculator further includes a measurer for measuring a diameter of a tumour or other body mass.

20. The calculator of claim 19, wherein the measurer is provided by a side of the calculator, which is graduated in units of length.

21. The calculator of claim 20, wherein the graduations are centimetres and millimetres.

22. The calculator of claim 17, wherein the calculator is rectangular or substantially rectangular in shape.

23. The calculator of claim 17, wherein the calculator is portable and configured to be hand held.

24. The calculator of claim 1, wherein the keys of the calculator are arranged in at least two banks, each bank including keys relating to a distinct or different part of the calculation process.

25. The calculator of claim 17, wherein the calculator includes a display, preferably an LCD screen.

26. (canceled)

27. The use of the calculator of claim 17, wherein to calculate the volume of a tumour or other body mass based on measurements taken from an image of the tumour or body mass.

28.-29. (canceled)
